(19) United States
(12) Patent Application Publication
(10) Pub. No.: US 2019/0162734 A1
(43) Pub. Date: May 30, 2019

(54) SYSTEM AND METHOD FOR LASER DESORPTION IONIZATION-MASS SPECTROMETRY ANALYSIS OF BIOMOLECULES IN CELLULAR COMPARTMENTS CAPTURED ON FUNCTIONALIZED SILICON NANOPOST ARRAYS

(71) Applicant: The George Washington University

(72) Inventors: Akos VERTES, Reston, VA (US); Andrew KORTE, Silver Spring, MD (US); Brian DAVIS, Niskayuna, NY (US); Sean DINN, Niskayuna, NY (US)

(21) Appl. No.: 16/304,937

(22) PCT Filed: May 26, 2017
(86) PCT No.: PCT/US17/34778

§ 371 Date: Nov. 27, 2018

Related U.S. Application Data
(60) Provisional application No. 62/342,418, filed on May 27, 2016.

(51) Int. Cl. G01N 33/68; C12Q 1/6872; H01J 49/04; H01J 49/16

(52) U.S. Cl. CPC G01N 33/6851; H01J 49/164; H01J 49/0418; C12Q 1/6872

(57) ABSTRACT

A system and method of performing mass spectrometry analysis on a sample such as a cellular component subject to laser desorption ionization is disclosed. A cell sample is lysed. An antibody is attached to a columnar array. The lysed cell sample for analysis is placed on the columnar array. The antibody retains components of the cell sample on the array to selectively capture cellular components of the cell sample. A laser is used to desorb and ionize the components of the cell sample. Mass spectrometry is performed on the ionized cell components.

### PRIORITY

The present application claims priority to U.S. Provisional Application No. 62/342,418 filed May 27, 2016, which is hereby incorporated by reference in its entirety.

### GOVERNMENT LICENSE RIGHTS

The subject matter of this application was made with support from the United States Government under a contract awarded by the Defense Advanced Research Projects Agency, Contract No. W911NF-14-2-0014000. The United States Government has certain rights in the invention.

### COPYRIGHT

A portion of the disclosure of this patent document contains material that is subject to copyright protection. The copyright owner has no objection to the facsimile reproduction by anyone of the patent disclosure, as it appears in the Patent and Trademark Office patent files or records, but otherwise reserves all copyright rights whatsoever.

### TECHNICAL FIELD

The present invention relates generally to analysis of materials in cellular components and specifically cells and cellular components captured on functionalized silicon nanopost arrays for mass spectrometry.

### BACKGROUND

The use of mass spectrometry for analysis of captured material provides high chemical specificity and wide applicability to many biomolecules without the need for molecular labeling. However, current capture technologies require further processing to detach and/or lyse nuclei or other cellular components before mass spectrometric analysis of the contents of captured cells or cell compartments can be attempted.

Silicon nanopost arrays (NAPA) have previously been developed as a substrate for ultrasensitive laser desorption ionization mass spectrometry (LDI-MS). The application of NAPA chips for LDI-MS of a wide variety of samples and chemical compounds has previously been demonstrated. NAPA-LDI-MS substrates invented by the Vertes Group have been commercialized by Protea Biosciences, Inc. under the trade name REDIchip®. The NAPA-LDI-MS technique involves deposition of a sample onto the nanopost array and subsequent irradiation with a focused laser beam. These nanopost arrays can also be functionalized with antibodies or other conjugates for selective capture of different cell compartments or intact cells, permitting selective analysis of biomolecules with high biological specificity. For example, recent developments have allowed for the selective capture of circulating tumor cells from biological fluids using microfluidics and antibody-functionalized nanopillars. Analysis of these cells, however, was not performed. The direct mass spectrometric analysis of biomolecules from these and other cells and cell compartments has the potential to benefit clinical diagnostics, for significant insight into the underlying biology of disease, drug/toxin effects, and normal cellular processes.

Thus, there is a need for a method of capturing and analyzing cells and compartments of cells directly from surfaces for mass spectrometry. There is also a need for a system that minimizes processing steps to reduce degradation of biomolecules in cellular components for mass spectrometry. There is also a need for a system for minimizing steps in analysis of cellular components in order to prevent the introduction of interferences.

### SUMMARY

According to one example, a system to analyze a biomolecular sample is disclosed. The system includes a columnar array having a plurality of columnar members. An antibody is attached to the columnar array. A laser is focused at a sample captured to the antibody on the columnar array. The laser is operable to apply a pulse to desorb and ionize the sample. The system includes a mass spectrometer. A controller is coupled to the mass spectrometer to analyze the output based on the detected ionized component from the columnar array.

Another example is a method to analyze a biomolecular sample. An antibody is attached to a columnar array. A sample for analysis is placed on the columnar array so the antibody selectively captures the sample. A laser is activated to desorb and ionize the sample. Mass spectrometry is performed on the ionized sample.

Another example is a laser desorption ionization mass spectrometry system for analyzing biomolecules. The system includes a silicon nanopost array having a plurality of nanoposts. An antibody against NUP98 is attached to the columnar array. A laser is focused at a cellular component sample obtained by a lysed cell. The cellular component sample is captured by the antibody on the nanopost array. The laser is operable to apply a pulse to desorb and ionize the cellular component sample. The system includes a mass spectrometer. A controller is coupled to the mass spectrometer to analyze the output based on the detected ionized cellular component sample captured on the nanopost array.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a block diagram of a laser desorption ionization-mass spectrometry system to analyze biomolecules in a sample captured on a columnar array;

FIG. 2A is an SEM image of the nanoposts in a nanopost array structure;

FIG. 2B is a schematic of the laser beam interaction with the columns of the nanopost array in FIG. 1;

FIG. 2C is an image of a set of nanopost arrays with cellular materials applied to the arrays;

FIG. 2D is an image of one of the nanopost arrays in FIG. 3B showing potential cellular components separated from the cell;

FIG. 3A is an AFM image of the microcolumns of a LISMA columnar array;

FIG. 3B is an SEM image of the cross-section view the LISMA array in FIG. 3A;

FIG. 3C is a two dimensional FFT of the nanopost array in FIG. 3A;

FIGS. 4A-4B are bright field images of cellular components captured on a nanopost array similar to that shown in FIG. 2A;

FIGS. 5A-5D are bright field images of cellular components after a cell sample has been subjected to different types of lysing;

FIGS. 6A-6D are bright field images of cellular components at a higher magnification after a cell sample has been subjected to different types of lysing;

FIGS. 7A-7D are SEM images of material from chemically lysed cellular samples captured on nanopost arrays;

FIGS. 8A-8D are SEM images of material from chemically lysed cellular samples captured on nanopost arrays;

FIGS. 9A-9D are SEM images of material electrically lysed cellular samples captured on nanopost arrays;

FIGS. 10A-10D are SEM images of material from glycerol lysed cellular samples captured on nanopost arrays;

FIGS. 11A-11B are SEM images of components of cellular samples after being applied to the nanopost array and interrogated by a laser;

FIG. 12A-12F shows NAPA-LDI mass spectra obtained from cellular samples on a nanopost array chip after different lysing techniques are used to separate the cellular components;

FIG. 13 show mass spectra over different regions of an interrogated nanopost array with captured cellular material;

FIG. 14 shows false-color images showing the distribution of metabolite ion signals over the surface of a nanopost array with captured cellular material in accordance with the operation of the system in FIG. 1;

FIG. 15A-15D shows fluorescent images of captured cellular material in accordance with the operation of the system in FIG. 1; and

FIG. 16 is a flow diagram of the process of analyzing cellular components by operation of the system in FIG. 1.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

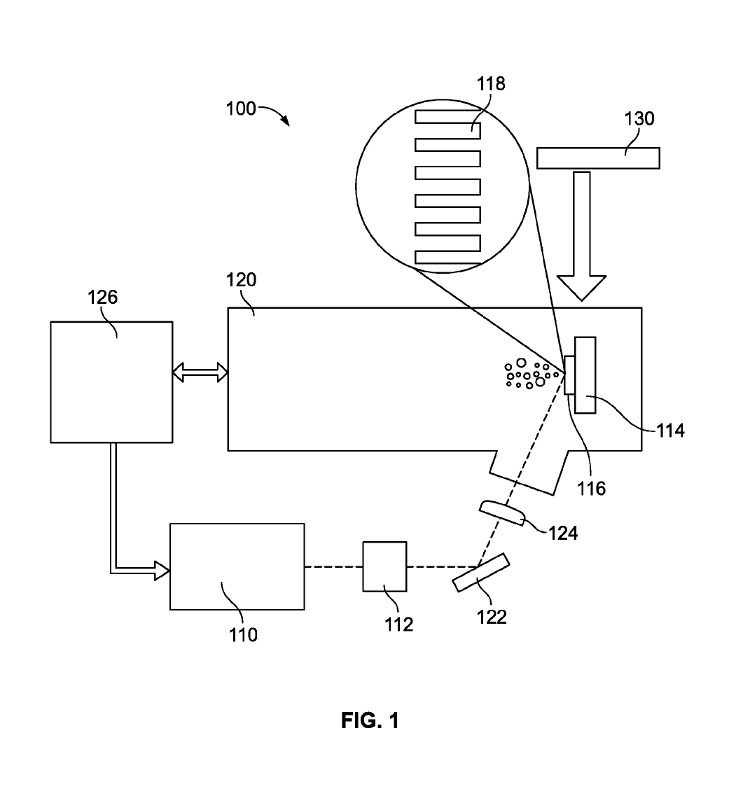

FIG. 1 is a block diagram of an example of a laser desorption ionization-based cellular sample measurement system 100. The system 100 includes a pulsed desorption laser 110 that emits light that is optionally polarized by a polarizer 112. The system 100 includes a sample stage 114 to which is affixed a columnar array such as a silicon nanopost array 116 that may be a NAPA chip. A sample is deposited on the nanopost array 116 and captured. An array of columnar members that may be nano-structures such as nanoposts 118 on the nanopost array 116 holds components of a cellular sample within a mass spectrometer 120. The system 100 includes optical components such as a mirror 122 and a focusing lens 124. A controller 126 is coupled to the mass spectrometer 120 and the laser 110 to operate the system and analyze the spectra output based on the detected ionized component from the nanopost array 116. The controller 126 may be used to control the power and duration of the pulses of emitted by the laser 110 and the spectra output from the mass spectrometer 120. An optional electrical lysing chip 130 may be provided to electrically lyse cellular samples to separate cellular components for application to the array 116.

The nanopost array 116 and sample stage 114 are arranged relative to the laser 110 so it is exposed to pulsed laser light at a predetermined angle. The laser light emitted from the pulsed laser 110 may contain a component polarized parallel to the columnar orientation, either as a result of the laser design or by introduction of the polarizer 112, which may be a Glan-Taylor calcite polarizer. However, the polarizer may 112 be any type of polarizer which allows for plane polarization of light from the pulsed laser source, as will be recognized by one of skill in the art. The laser light is focused onto the nanopost array 116 by the focusing lens 124. Absorption of the incident laser energy by the nanoposts 118 leads to heating and desorption/ionization of deposited material, generating gas-phase ions for analysis by the mass spectrometry system 120.

The system 100 allows the efficient mass spectrometry analysis of samples such as cells or cellular components such as nuclei. In this example, cellular samples may be first partially lysed in order to isolate the desired components for analysis. The lysis is applied to disrupt the outer plasma membrane of the cell, releasing internal organelles like mitochondria and nuclei. Cell lysis may be performed by a number of methods, including chemical techniques or electrical lysis performed by the optional lysing chip 130. Antibodies are chemically conjugated to the surface of the nanoposts 118. The antibodies are selected to capture components of interest on the nanoposts 118.

The system 100 allows for the conjugation of any antibody onto the surface of the nanoposts 118 to facilitate specific capture of a target cell type or organelle. Of significant interest are antibodies that interact with outer membrane or surface proteins on the target cell or organelle. For example, an antibody against the Nup98 nuclear pore complex protein may be used to capture nuclei, however there are many other antibody options for capturing nuclei that those skilled in the art will acknowledge. These options include but are not limited to antibodies for other nuclear pore proteins and/or nesprins. Other organelles could be captured using antibodies against proteins present on the membrane of those organelles including but not limited to Tom22 for mitochondria. Similarly, specific attachment of cell types could be achieved based on specific protein expression. Non-limiting examples of antibodies for specific cell capture includes CD3 for T cells, CD19 for B cells, CD34 for hematopoietic stem cells and CD90/EpCAM for circulating tumor cells.

In this example, cellular components are applied on the array of nanostructures 118 and the cellular components are captured by the formation of antibody-protein complexes. The pulsed laser 110 is activated to heat the nanoposts 118 in the array 116 resulting in desorption and ionization of a portion of the captured material. The mass spectrometer 120 is then used to output spectra for chemical analysis of the ionized components. Alternatively, the system 100 may be used to analyze other samples such as whole cells (without lysis) or specific subcellular features, such as a thin membrane at the edge of the cell (lamellipodia), or for microbial cells. Analysis of individual yeast cells has been demonstrated previously.

The production and use of microcolumn and nanocolumn arrays that harvest light from a laser pulse to produce ions is described herein. The systems described seem to behave like a periodic antenna arrays with ion yields that show dependence on the plane of laser light polarization and the angle of incidence. These photonic ion sources enable an enhanced control of ion production on a micro/nano scale and its direct integration with miniaturized analytical devices.

In the system 100, the columnar array for the analysis of a sample by mass spectrometry may be a nanopost array (NAPA) or a laser-induced silicon microcolumn array (LISMA). As will be detailed below, the nanopost array results in more uniformly placed columns of more uniform height than the columns of a LISMA. Thus, use of a nanopost array such as the nanoposts 118 for the system 100 is preferred.

In one example, the NAPA chips that may be used for the array 116 in FIG. 1 may be produced with silicon wafers. Fabrication of the silicon nanoposts may be achieved using deep ultraviolet projection photolithography (DUV-PL) and deep reactive ion etching (DRIE). Low resistivity (0.001-0.005 ohm-cm) <100> p-type silicon wafers (Silicon Valley Microelectronics, Inc., Santa Clara, Calif.) may be used. An anti-reflective coating (AR2-600, DOW Shipley, Marlborough, Mass.) may be spin coated on the native silicon surface at 3500 rpm for 30 s and baked on a hot plate at 220° C. for 1 min. Negative-tone DUV photoresist (UVN-2300, DOW Shipley, Marlborough, Mass.) may then be spin-coated at 3500 rpm for 30 s before a bake at 110° C. for 90 s. A 248 nm wavelength lithography stepper system (PAS 5500/300, ASML, Veldhoven, Netherlands) may be used for pattern transfer. After exposure, a 60 s hot plate bake is performed at 105° C. The photoresist is developed using an agitated bath of AZ300MIF (Clariant, Somerville, N.J.) for 60 s, followed by a bath in deionized water for 60 s. The wafers are dried using N₂ gas. The DRIE (PlasmaTherm 790, St. Petersburg, Fla.) and a vertical Si etch was performed with a chamber pressure of 19 mTorr, an ICP power of 825 W, and an RIE power of 15 W for 9 min. The mixture of etchant gases may be as follows: C₄F₈ (52 sccm), SF₆ (28 sccm), and Ar (20 sccm). Wafers were etched to a depth of 1100 nm. After DRIE, wafers were cleaned using 02 plasma (Technics PEII, Pleasanton, Calif.) at 300 mTorr and 100 W for 3 min. Final post dimensions may be 150 nm in diameter, 1100 nm in height with a periodicity of 337 nm. The nanopost arrays are stored in a low humidity environment until use.

Alternatively, the nanoposts may be fabricated on the silicon wafers by metal assisted chemical etching. The silicon surface (Si (100), B-doped, 0.004-0.007 Ωcm) of the wafer may be cleaned by a RCA-I approach. Nanospheres such as polystyrene nanopsheres with diameters of 1.39 um, 622 nm and 390 nm may be deposited as a monolayer for lithographic structuring. The nanospheres may be etched in O2 plasma to reduce their diameters. Silver is evaporated onto the surface of the silicon. The nanospheres are lifted off and the wafers are rinsed. This process creates a metal film with openings correlating with the size of the etched nanospheres. The wafer is then etched and the metal film sinks into the silicon, which is solved by the etching solution. The chips are then rinsed with nitric acid to remove the silver and nanoposts are obtained equal to the size of the openings in the metal film.

Examples of LISMA, which may be used in the above process, may be found in U.S. Patent Publication No. 2009/0321626, which is hereby incorporated by reference herein. The arrays may be adapted to be in cooperative association with a polarized desorption laser beam having a specific wavelength. The microcolumn array is typically a silicon wafer made from low resistivity p-type or n-type silicon having a plurality of about 100 μm² to 1 cm² processed areas that are covered with quasi-periodic columnar structures. The structures are generally aligned perpendicular to the silicon wafer but they may also be aligned at other well defined angles. The structures generally have dimensions according to the laser used in the desorption of a sample for mass spectrometry analysis. For example, the columnar structures may have a height of about 1 to 5 times the wavelength of the desorption laser, a diameter equal to about one wavelength of the desorption laser, and a lateral periodicity of about 1.5 times the wavelength of the desorption. The columnar structures may have a height of 2 times the wavelength of the desorption laser.

The LISMA may be produced by processing a polished silicon wafer by exposing it to multiple ultrashort ultraviolet, visible or infrared laser pulses of about 50 femtoseconds to about 100 picoseconds duration in different processing environments, such as liquid water, sulfur hexafluoride, glycerol and aqueous solutions such as bases or acids. Particular examples of aqueous solutions that may be used include sodium hydroxide and acetic acid solutions. The use of different processing environments allows for the production of LISMA with different chemical residues in the columnar structures that may facilitate ionization and/or desorption. As a non-limiting example, use of sodium hydroxide processing environment provides a LISMA with sodium hydroxide residues and/or surface hydroxyl groups on the columnar structures that enhances ion production and desorption.

Where a LISMA are used as the substrate for capture and desorption, the laser used for processing the columnar array in the system 100 may be the same or different from the laser used during desorption of samples. It will be apparent to those of skill in the art that various types of lasers can be used in producing the arrays and for sample desorption, including gas lasers such as nitrogen and carbon dioxide lasers, and solid-state lasers, including lasers with solid-state crystals such as yttrium orthovanadate (YVO4), yttrium lithium fluoride (YLF) and yttrium aluminum garnet (YAG) and with dopants such as neodymium, ytterbium, holmium, thulium, and erbium. In certain embodiments of the present invention, the laser used for processing the arrays is a mode-locked Nd:YAG laser and the laser used for desorption of the sample is a nitrogen laser.

The array 116 may be made from other semiconducting materials, such as germanium, gallium arsenide and the like. The columnar arrays such as the array 116 used for the capture and desorption in this example may have columnar structures with a height of from about 200 nm to about 1500 nm, preferably about 1100 nm, a diameter of from about 200 nm to about 400 nm, preferably 150 nm, and a lateral periodicity of from about 450 nm to about 550 nm, preferably 337 nm. It is further contemplated that the arrays used may have columnar structures with other dimensions consistent with nanocolumn arrays and microcolumn arrays as are known in the art.

The system 100 may also comprise any laser desorption ionization-mass spectrometry system having: i) a micro- or nanostructure array for holding a sample; ii) a pulsed laser for irradiation of the sample, leading to desorption and ionization of deposited material; iii) focusing optics based on lenses, mirrors or optical fibers; iv) an optional polarizer for polarizing the laser radiation; and v) a mass spectrometer for analyzing the produced ions. The system 100 may also include a positioning apparatus and software for lateral positioning of multiple points on the micro- or nanostructured sample.

Irradiation from a pulsed laser such as the laser 110 in FIG. 1 is focused onto a photonic ion source comprised of an array of columnar nano- or microstructures after the sample such as a cellular component are deposited onto the surface of the array. The columnar shape of the nanostructures leads to absorption of laser radiation and produces molecular, and at sufficiently high laser fluences, fragment ions that can be separated and detected by a system such as the mass spectrometer 120 in FIG. 1. Production of ions from the surface can be modulated by varying parameters such as laser fluence, angle of incidence, and plane of polarization of the incident beam.

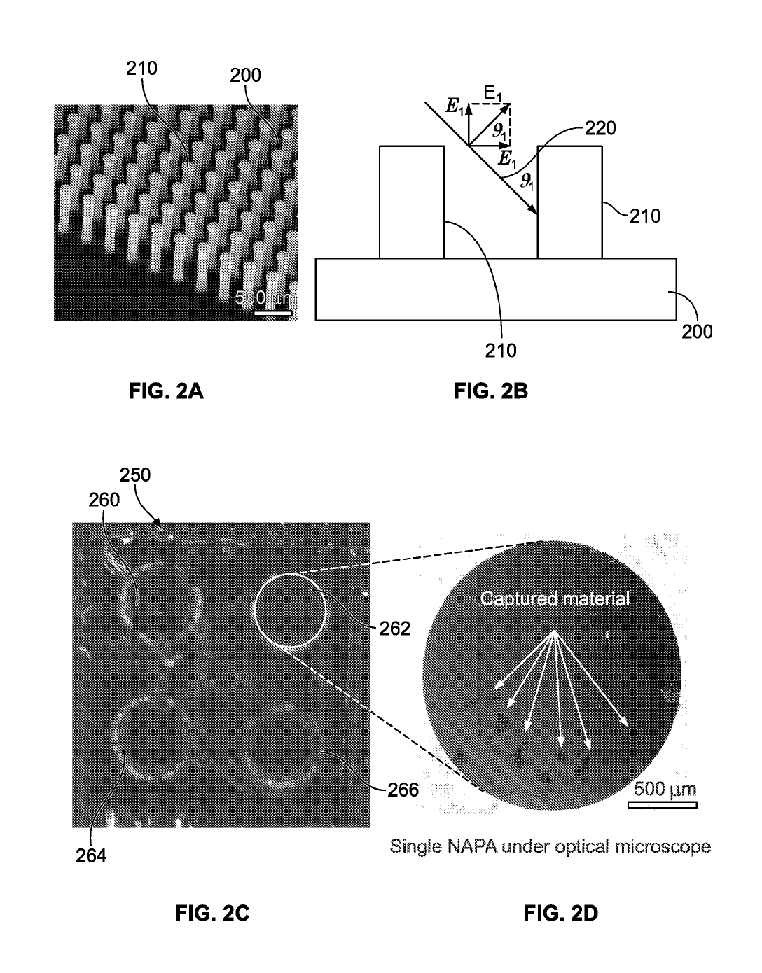

FIG. 2A is a scanning electron microscope (SEM) image of a NAPA structure 200 that reveals the periodic arrangement of microcolumns 210 in a NAPA that may be used for the columnar array 116 in FIG. 1.

FIG. 2B is a schematic of the interaction between incident laser energy and the columns in a nanopost or microcolumn array such as the nanopost structure 200. As shown in FIGS. 2A and 2B, the structure 200 includes nanoposts such as the microcolumns 210. The laser beam shown as an arrow 220 is focused on the microcolumns 210 at a predetermined angle. As explained above, the laser pulses desorb and ionize sample cellular components captured by antibodies conjugated to the microcolumns 210.

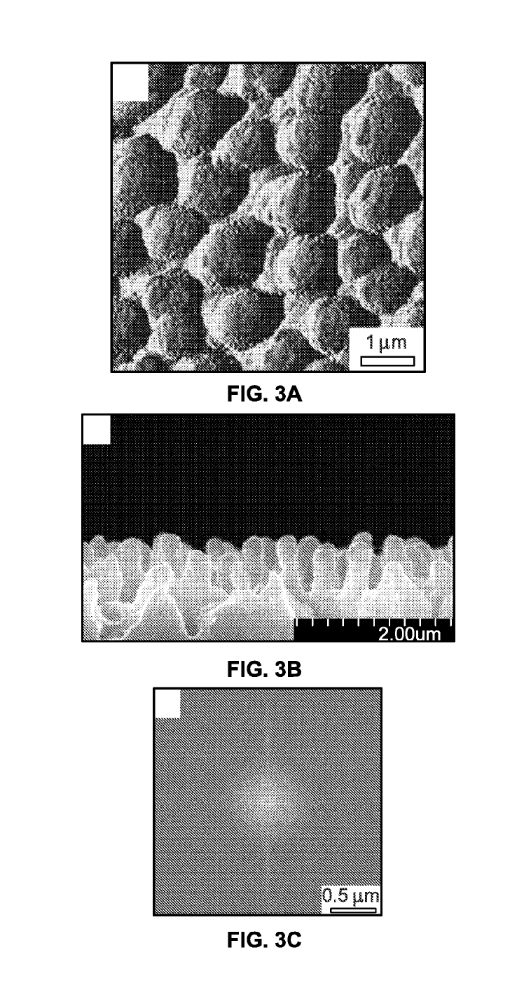

FIG. 2C is an image of an example NAPA chip 250 that includes four nanopost arrays 260, 262, 264 and 266 after deposition of isolated cellular nuclei. The example NAPA chip 250 may be used for the array 116 in FIG. 1. The arrays 260, 262, 264 and 266 each include millions of nanoposts. The nanoposts in each of the arrays 260, 262, 264 and 266 in FIG. 2C and the structure 200 in FIG. 2A are of a more uniform height and diameter and have a more uniform distribution than the LISMA shown below in FIG. 3A-2C. The moderate-resolution image in FIG. 2C was taken using the integrated camera in a matrix-assisted laser desorption ionization (MALDI) mass spectrometer system that could be used for the mass spectrometer 120 in FIG. 1.

FIG. 2D is an image from an optical microscope of one of the nanopost arrays 262 of the chip 250 that shows the captured material from the cellular sample. The partial coverage of the nanopost array 262 with nuclei observed in FIG. 2D is useful for distinguishing signals arising from captured material from background signals. The nanopost arrays 262 in this example were functionalized with anti-Nup98 in this example to bind nuclei. Nuclei were isolated from human hepatocellular carcinoma cells (cell line HepG2/C3A) using a commercial subcellular fractionation kit (ATTO Corp. #WSE-7422). Briefly, HepG2/C3A cells were chemically lysed and centrifuged to isolate nuclei. Nuclei were resuspended in PBS pH 7.4 and allowed to deposit onto the functionalized NAPA surface on the array 262 at room temperature for 15 minutes. After deposition, the surface was washed with PBS to remove unbound material and dried under a gentle stream of nitrogen. Optical images were acquired prior to the NAPA-MS analysis.

Although the example NAPA chip 250 in FIG. 2C includes four nanopost arrays, other numbers of arrays may be used. For example, a microscope slide sized wafer suitable for cellular samples or components of cellular samples may include 96 arrays for large-scale analyses.

FIG. 3A is a top view of an example LISMA by an atomic force microscope (AFM) that reveals the quasi-periodic arrangement of the microcolumns in a LISMA that may be used for the columnar array instead of the nanopost array 116 in FIG. 1. FIG. 3B is a cross sectional SEM image that shows an average column height and diameter of about 600 nm and about 300 nm, respectively, with about 200 nm troughs between the columns in the example LISMA. FIG. 3C is a two-dimensional FFT of a top view SEM image which reveals the about 500 nm mean periodicity of the LISMA structures.

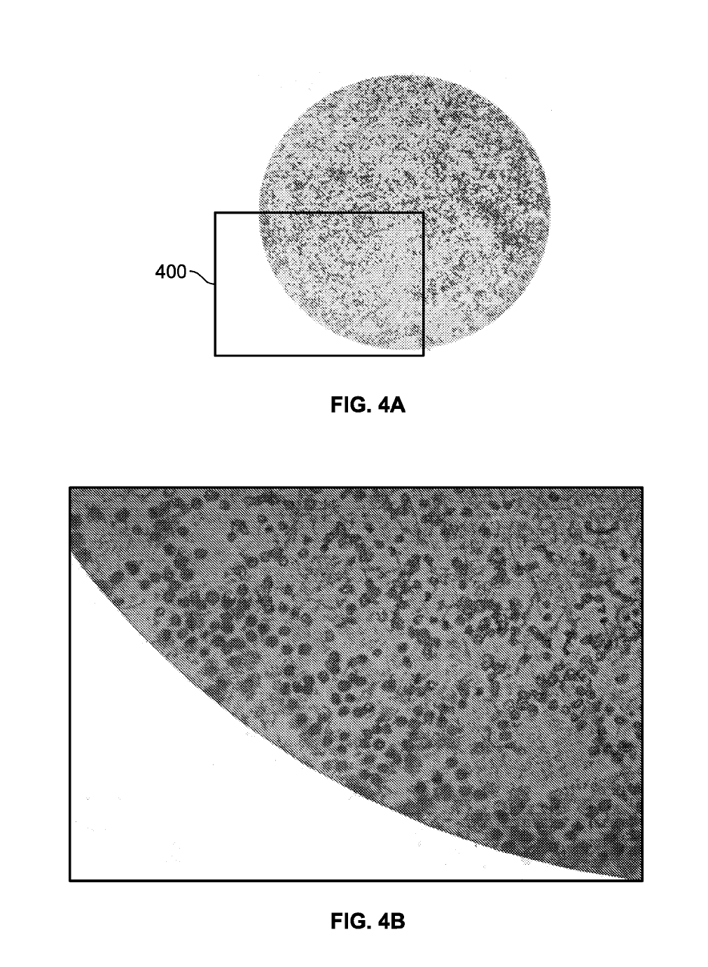

FIG. 4A and FIG. 4B are bright field microscope images at 50× and 100× magnification, respectively, of HepG2/C3A cellular components captured on a nanopost array using the above described procedure prior to pulsing the laser 110 in FIG. 1. Here, further optimization of capture and washing protocols has resulted in more uniform coverage of the NAPA surface with captured material. Various irregular shapes and round shapes are components from the cellular sample after lysing. The round shapes are possible nuclei that may be of interest for mass spectrometry analysis.

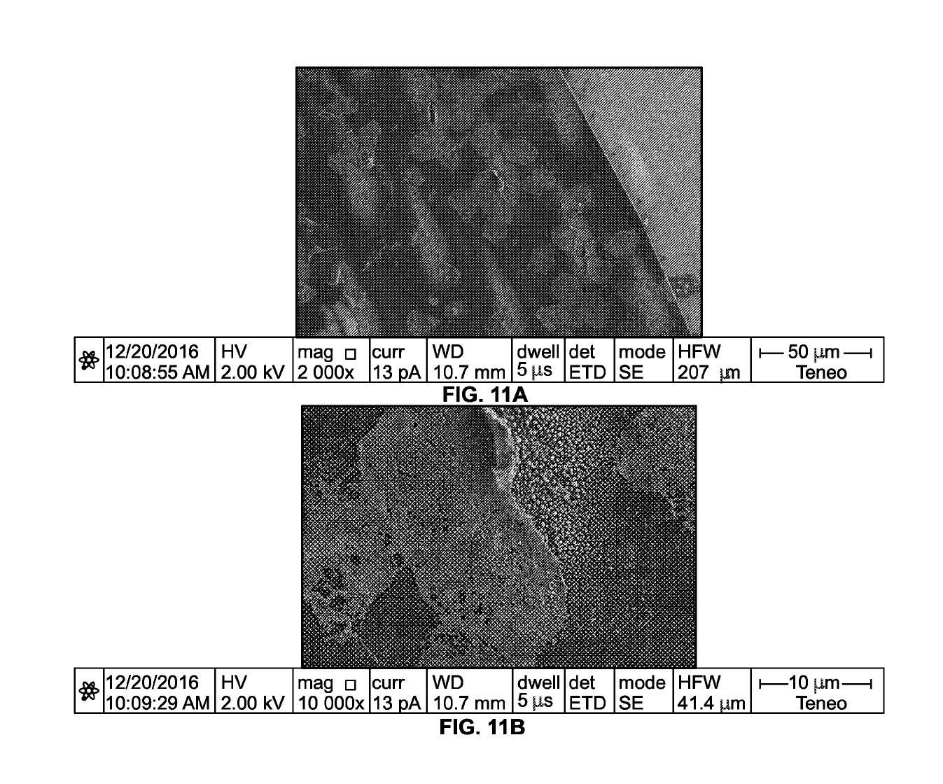

FIGS. 3-10 show images obtained after capture of cellular material but prior to interrogation of the surface with the laser. FIGS. 11A-11B shows images taken after the cellular components on the nanopost array were irradiated and mass spectrometry analysis was performed.

A number of different lysis techniques can be used to obtain cellular components for capture onto micro- or nanostructured surfaces. The different lysis techniques have been demonstrated including chemical lysis, electrical lysis and application of glycerol.

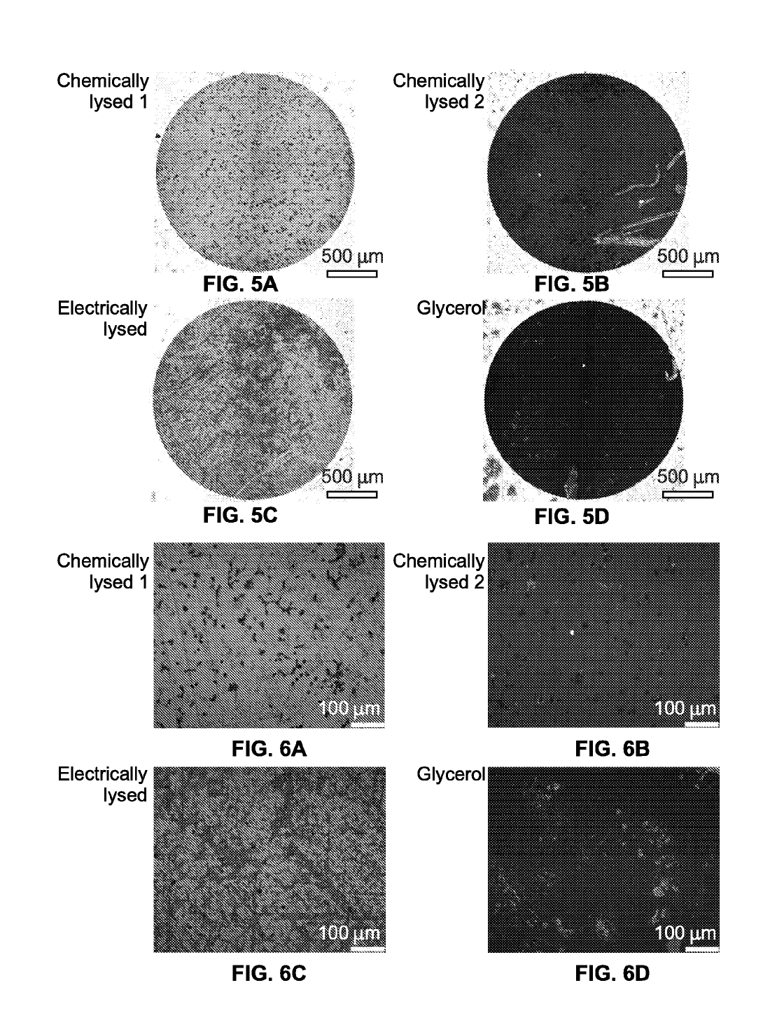

FIGS. 5A-5D and 6A-6D are bright field images of the cellular samples after the different lysing techniques are used. Thus, FIG. 5A and FIG. 6A are bright field image of cellular materials after a first trial of a chemical lysing process. Specifically, the chemical lysing process in this example involved lysing HepG2/C3A cells for 10 minutes using the detergents supplied in the Atto EZ Subcell fractionation kit (WSE-7422). The cells were passed through a 1 mL pipette tip 25 times then were centrifuged at 700 g for 10 minutes to pellet the nuclei. The nuclei were resuspended in PBS pH7.4 and applied onto the NAPA array such as the array 116. The NAPA chip was washed in PBS or 1:10 diluted PBS to eliminate unbound material.

FIG. 5A is a bright field image at 50× magnification while FIG. 6A is a bright field image at 100× magnification. FIG. 5B and FIG. 6B are bright field images of another trial of chemically lysed cellular materials using the above described chemical lysing process. FIG. 5B is an image at 50× magnification while FIG. 6B is an image at 100× magnification.

FIG. 5C and FIG. 6C are bright field image of electrically lysed cellular materials. FIG. 5C is a bright field image at 50× magnification while FIG. 6C is a bright field image at 100× magnification. In this example, HepG2/C3A cells in PBS were passed through an electrical lysis chip such as the chip 130 in FIG. 1 at 200 uL/min and lysed using 100V applied voltage. Lysed cell material was collected in an Eppendorf tube, kept on ice until attachment onto the NAPA chip. The chip was washed in 1:10 PBS to eliminate unbound material. In the system 100 in FIG. 1, an optional channel may be used to move the lysed samples from the electrical lysis chip 130 to the nanopost array 116.

FIG. 5D and FIG. 6D are bright field images of glycerol lysed cellular materials. FIG. 5D is an image at 50× magnification while FIG. 6D is an image at 100× magnification.

As may be seen, the lysing methods of chemical lysing and electrical lysing result in the best separation of nuclei from the cellular components as shown in FIGS. 5B and 6B for chemical lysing and as shown in the images in FIGS. 5C and 6C for electrical lysing.

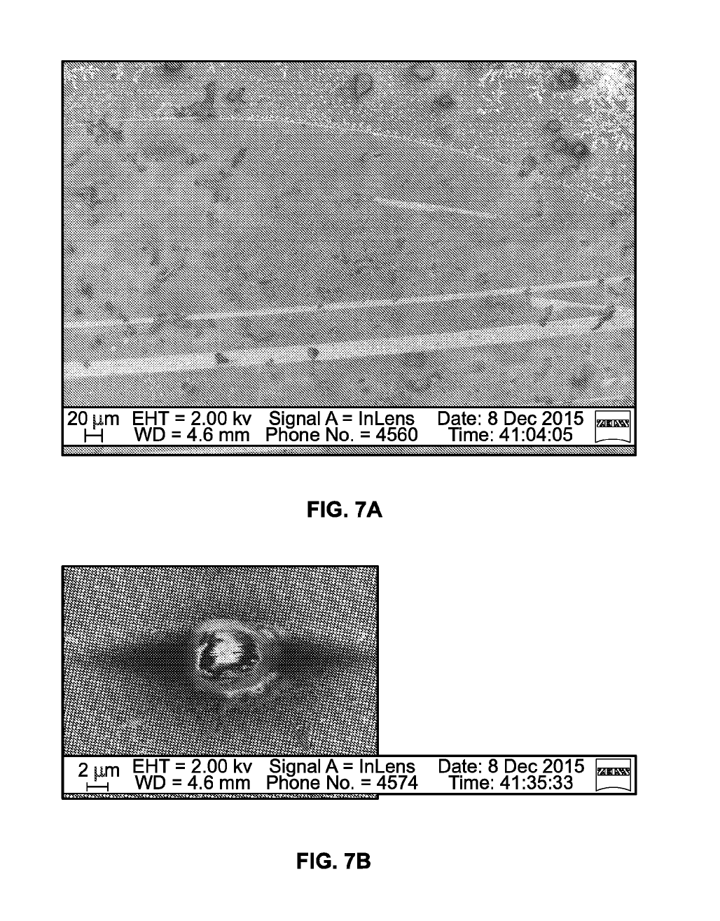

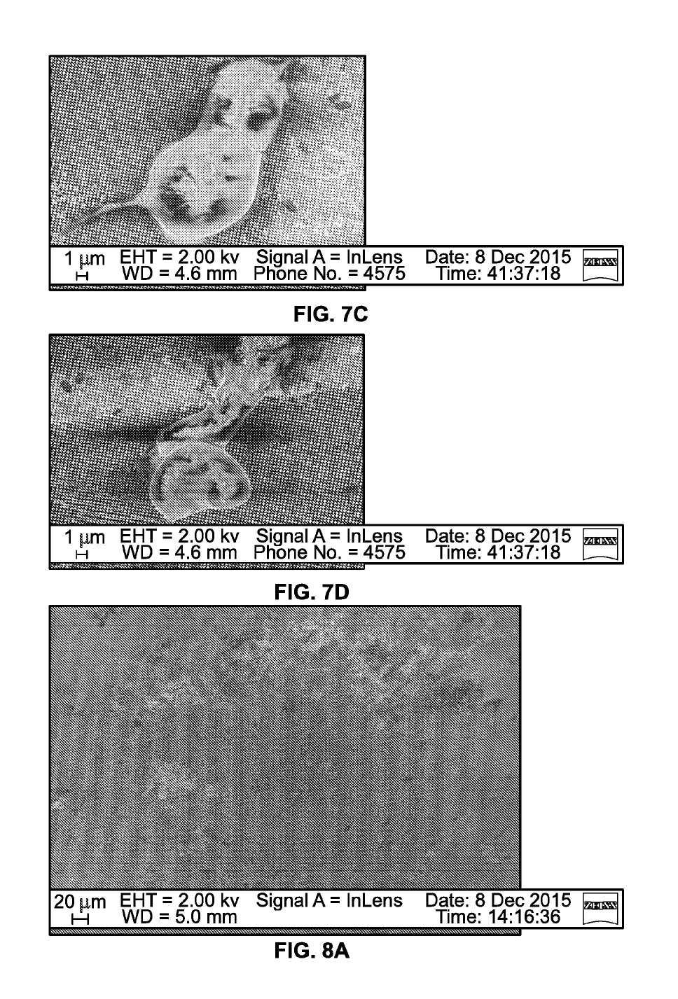

FIG. 7A-7D are SEM images of cellular materials captured following chemical lysis as explained above. FIG. 7A is an SEM image that shows different potential components of interest after lysis. The captured components may include remnants of the cell structure, mitochondria, nuclei and cell membrane fragments. The SEM image in FIG. 7A was taken under 500× magnification. The accelerating voltage was 2 kV, the extractor voltage was set at 4 kV, images were acquired with the in-lens detector, the extractor current was 359 μA, the filament current was 2.339A, the scan speed was 4, and noise reduction was performed by line integration with N=30. FIGS. 7B-7D are greater magnification SEM images that show different components of potential interest from the cellular sample. For example, the image in FIG. 7B shows a probable nucleus. The SEM images in FIGS. 7B-7D were taken under 10,000× magnification. The other conditions were similar to those of the image in FIG. 7A.

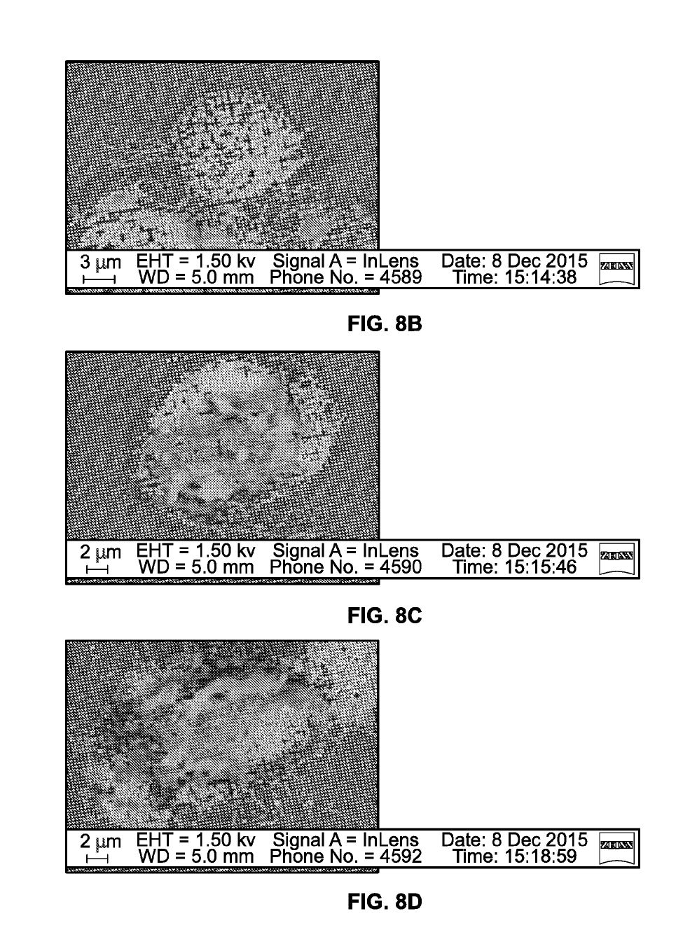

FIG. 8A-8D are SEM images of cellular materials captured following chemical lysis as explained above. FIG. 8A is an SEM image that shows different potential components of interest after lysing. The SEM image in FIG. 8A was taken under 500× magnification. The accelerating voltage was 2 kV, the extractor voltage was set at 4 kV, images were acquired with the in-lens detector, the extractor current was 359 μA, the filament current was 2.339A, the scan speed was 4, and noise reduction was performed by line integration with N=30. FIGS. 8B-8D are greater magnification SEM images that show different components of potential interest from the cellular sample. The SEM images in FIGS. 8B-8D were taken under 10,000× magnification. The other conditions were similar to those of the image in FIG. 8A.

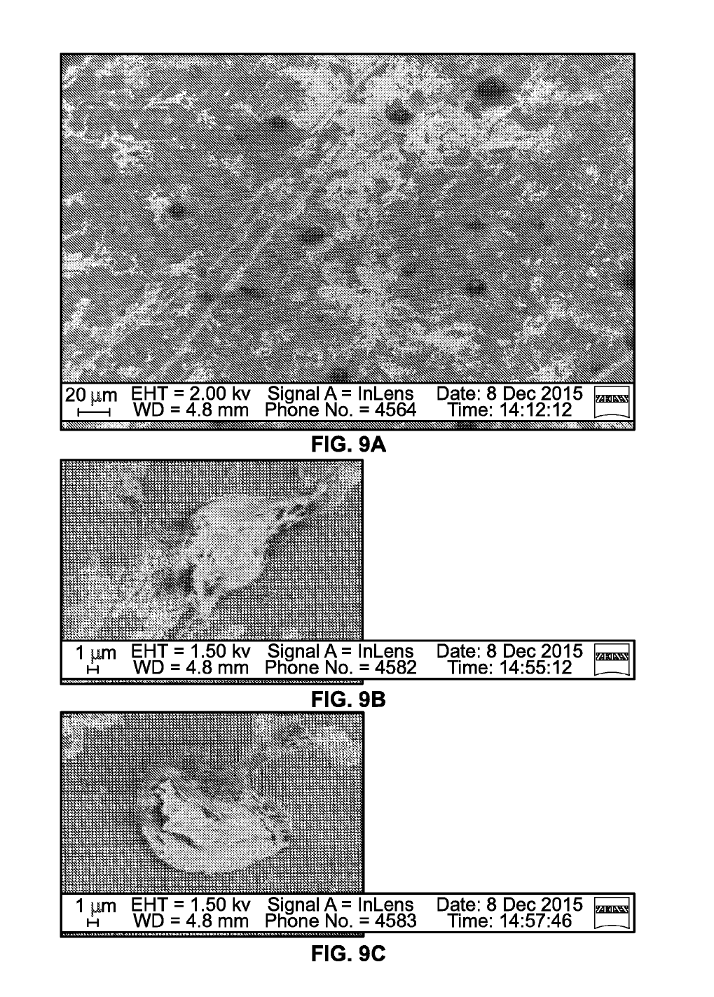

FIG. 9A-9D are SEM images of the cellular sample after being electrically lysed. FIG. 9A is an SEM image that shows different potential components of interest after lysing. The SEM image in FIG. 9A was taken under 500× magnification. The accelerating voltage was 2 kV, the extractor voltage was set at 4 kV, images were acquired with the in-lens detector, the extractor current was 359 μA, the filament current was 2.339A, the scan speed was 4, and noise reduction was performed by line integration with N=30. FIGS. 9B-9D are greater magnification SEM images that show different components of potential interest from the cellular sample. The SEM images in FIGS. 9B-9D were taken under 10,000× magnification. The other conditions were similar to those of the image in FIG. 9A.

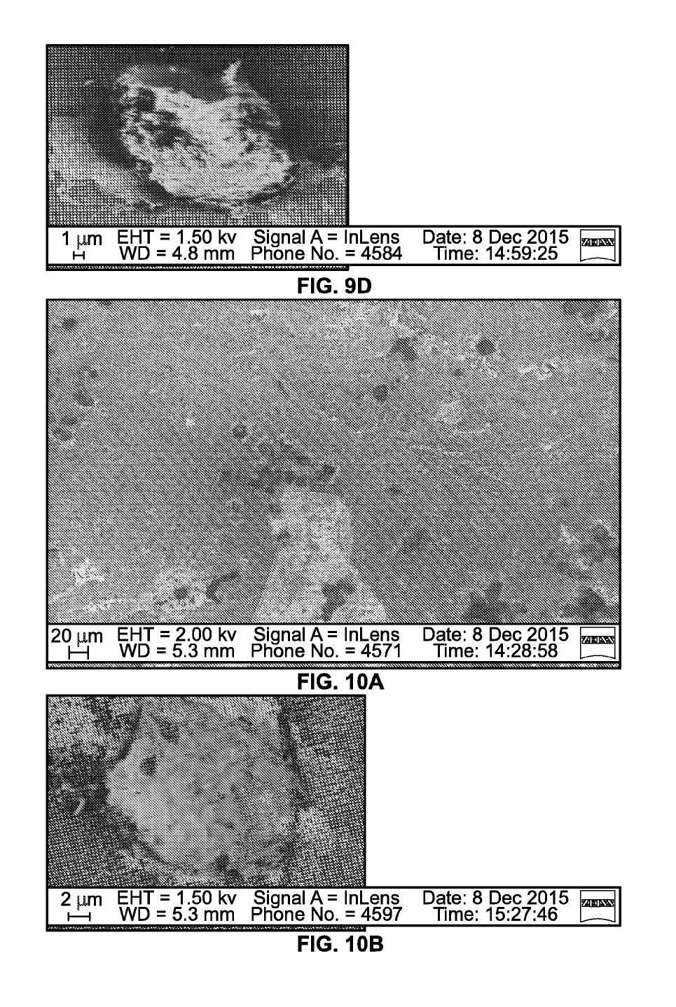

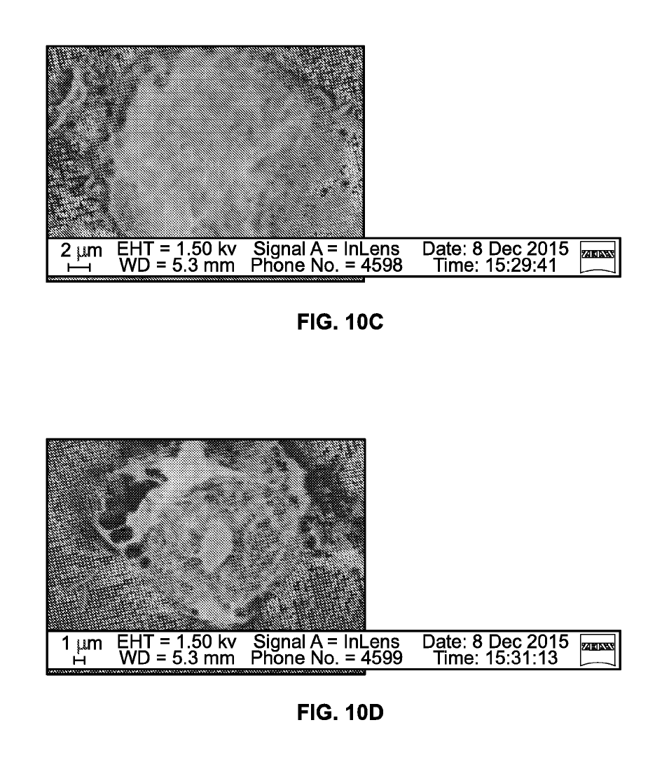

FIG. 10A-10D are SEM images of the cellular sample after being lysed by application of glycerol. FIG. 10A is an SEM image that shows different potential components of interest after lysing. The SEM image in FIG. 10A was taken under 500× magnification. The accelerating voltage was 2 kV, the extractor voltage was set at 4 kV, images were acquired with the in-lens detector, the extractor current was 359 μA, the filament current was 2.339A, the scan speed was 4, and noise reduction was performed by line integration with N=30. FIGS. 10B-10D are greater magnification SEM images that show different components of potential interest from the cellular sample. The SEM images in FIGS. 10B-10D were taken under 10,000× magnification. The other conditions were similar to those of the image in FIG. 10A.

As explained above, once a cellular sample is lysed and the isolated components are applied to the nanopost array 116 in FIG. 1, the laser 110 is pulsed to desorb and ionize the components on the nanoposts 118 on the array 116. FIG. 11A and FIG. 11B are SEM image of the nanopost array 116 after the laser 110 is pulsed. FIG. 11A is an SEM image at 2000× magnification and shows melted posts in regions sampled by the laser as evidenced by the regularly spaced lightly colored regions. FIG. 11B is an SEM image at 10,000× magnification and shows a sampling of captured material in laser-irradiated regions as indicated by deformation and removal of material.

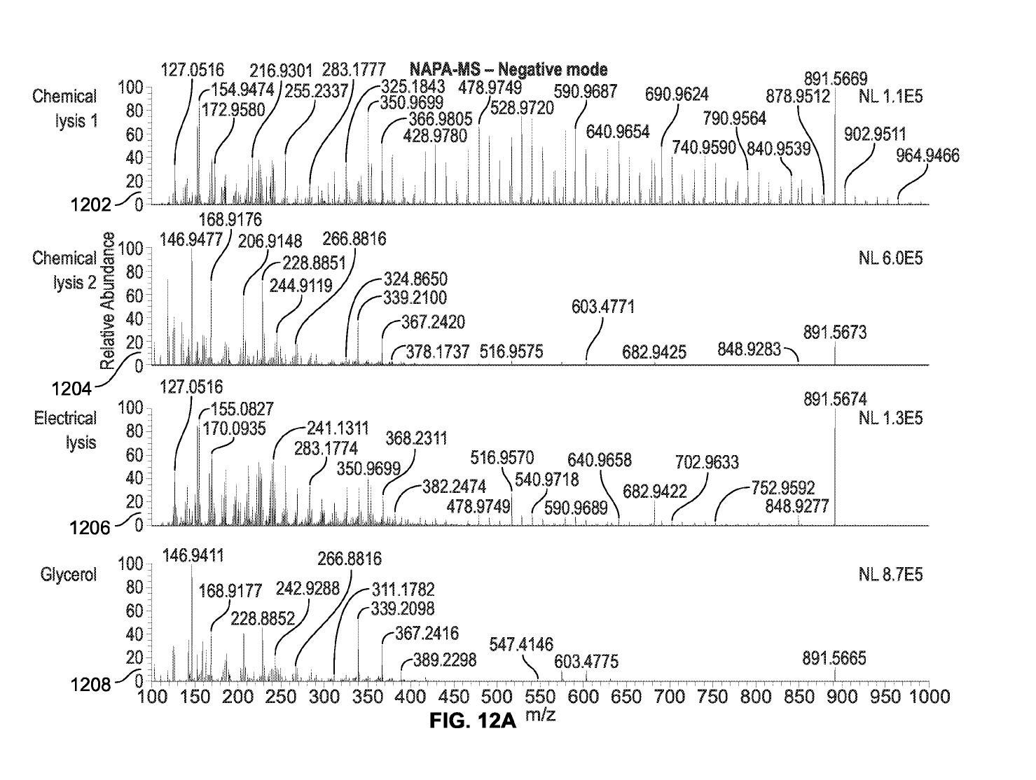

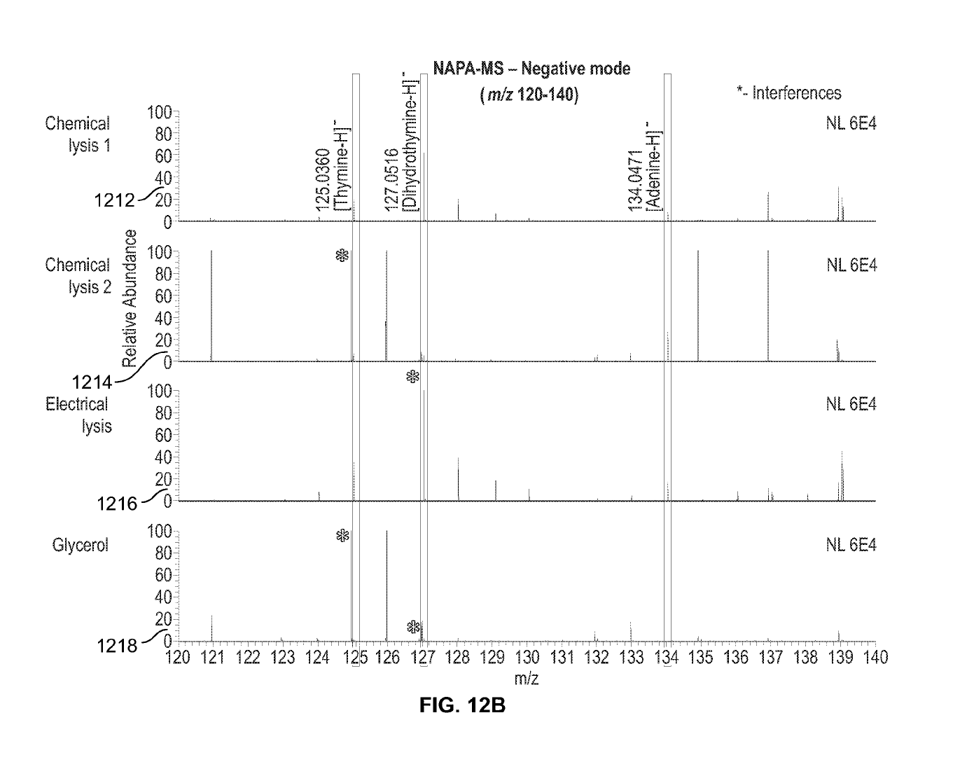

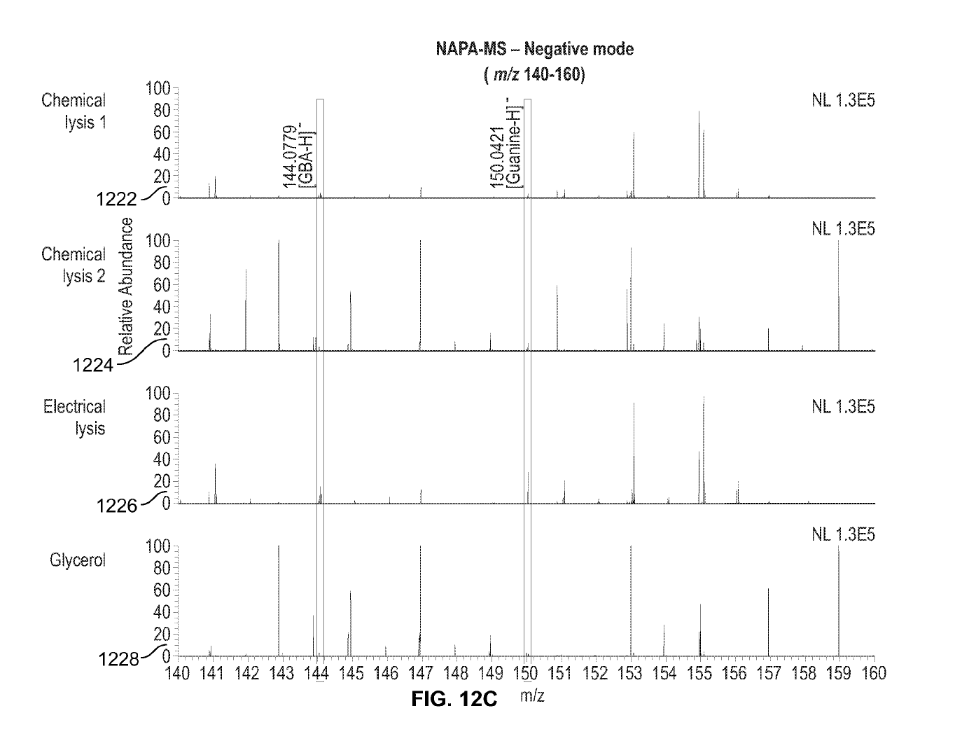

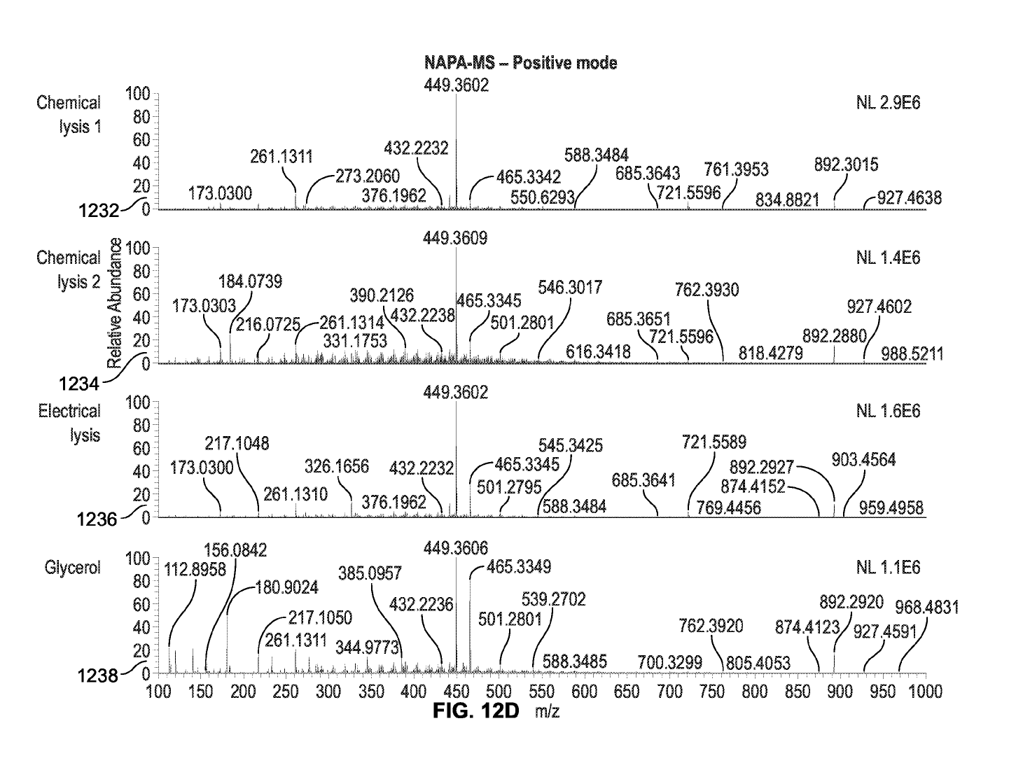

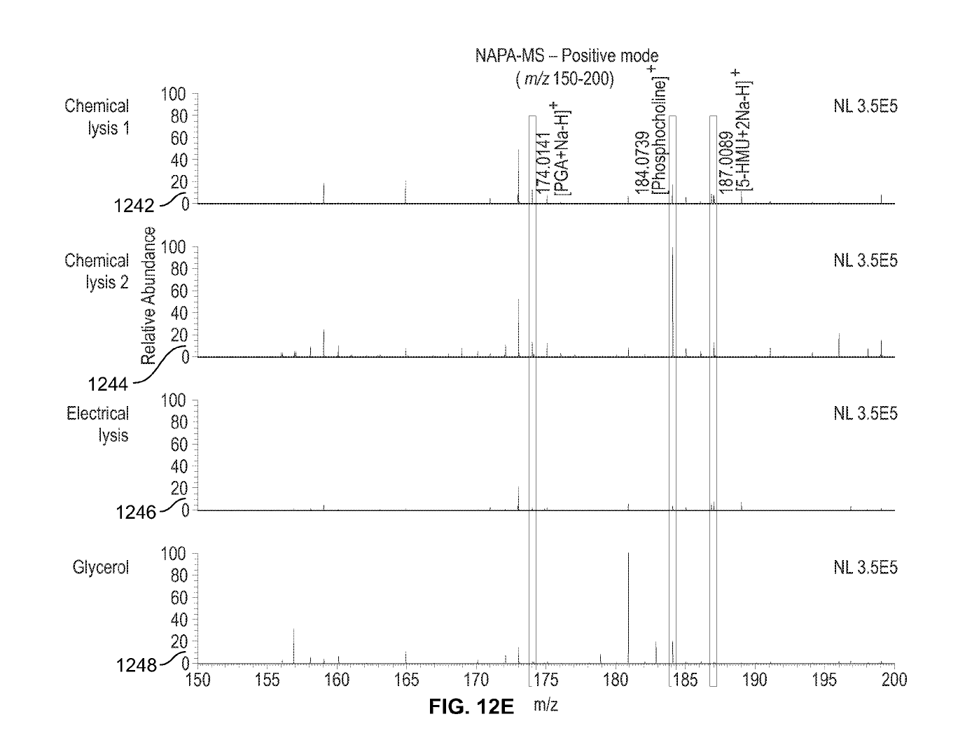

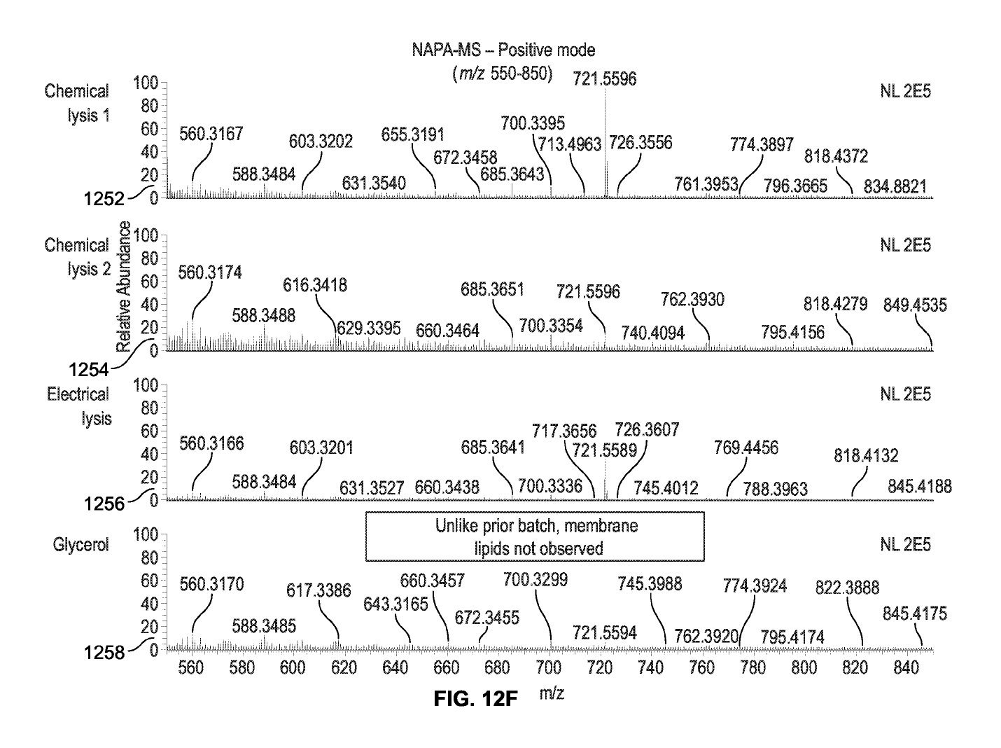

After the cellular components are applied to the nanopost array and the laser is pulsed to desorb and ionize the components, the mass spectrometer 120 in FIG. 1 is used for analysis of the ionized components. FIGS. 12A-12F show NAPA-LDI mass spectra obtained from the differently lysed cellular components described above. All spectra presented in FIGS. 12A-12F were acquired using a Thermo Scientific MALDI-LTQ-Orbitrap XL system in the Orbitrap mass analyzer at a nominal mass resolving power of 30,000. Nanopost surface areas were scanned in a rastering fashion using the imaging functionality of the instrument control software.

FIG. 12A shows four different mass spectra 1202, 1204, 1206 and 1208 from analysis of cellular components isolated by different lysis techniques. The spectra 1202 and 1204 were collected from captured components obtained by the chemical lysis procedure explained above. The spectrum 1206 was collected from captured cellular components obtained by the electrical lysis procedure explained above. The spectrum 1208 was collected from captured components obtained by the glycerol based lysis procedure explained above.

The spectra 1202, 1204, 1206 and 1208 were acquired in the negative ion mode. The output represented by the graph 1202 has an absolute signal intensity of 1.1E5. The output represented by the graph 1204 has an absolute signal intensity of 6.0E5. The output represented by the graph 1206 has an absolute signal intensity of 1.3E5. The output represented by the graph 1208 has an absolute signal intensity of 8.7E5. The laser power setting was 15 uJ with 3 shots/scan and 1 scan/step. The scan range was m/z 100-1500 and the raster step size was 50 μm. As may be seen in FIG. 12A, the first chemical lysis trial resulted in multiple sets of peaks that indicate the generation of salt ion clusters. The second chemical lysis trial and the electrical lysis outputs in spectra 1204 and 1206 resulted in peaks at the left and right of the graphs that are of interest in terms of analysis of cellular components such as cell nuclei.

FIG. 12B shows four different mass spectra 1212, 1214, 1216 and 1218 from analysis of cellular components isolated by different lysis techniques. The spectra 1212 and 1214 were collected from captured cellular components obtained by the chemical lysis procedure explained above. The spectrum 1216 was collected from captured cellular components obtained by the electrical lysis procedure explained above. The spectrum 1218 was collected from cellular components obtained by the glycerol based lysis procedure explained above.

The spectra 1212, 1214, 1216 and 1218 were acquired in the negative ion mode. The absolute signal intensity for the outputs in FIG. 12B was 6E4. The laser power setting was 15 uJ with 3 shots/scan and 1 scan/step. The scan range was m/z 120-140 and the raster step size was 50 um. As may be seen in FIG. 12B, peaks assigned as [Thymine-H]⁻, [Dihydrothymine-H]⁻ and [Adenine-H]⁻ were detected.

FIG. 12C shows four different mass spectra 1222, 1224, 1226 and 1228 from analysis of cellular components isolated by different lysis techniques. The spectra 1222 and 1224 were collected from captured cellular components obtained by the chemical lysis procedure explained above. The spectrum 1226 was collected from captured cellular components obtained by the electrical lysis procedure explained above. The spectrum 1228 was collected from captured cellular components obtained by the glycerol based lysis procedure explained above. The spectra 1222, 1224, 1226 and 1228 were acquired in the negative ion mode. The absolute signal intensity for the outputs in FIG. 12C was 1.3E5. The laser power setting was 15 uJ with 3 shots/scan and 1 scan/step. The scan range was m/z 140-160 and the raster step size was 50 um. As may be seen in FIG. 12C, peaks assigned as [4-Guanadinobutanoic acid (GBA)-H]⁻ and [Guanine-H]⁻ were discovered.

FIG. 12D shows four different mass spectra 1232, 1234, 1236 and 1238 from analysis of cellular components isolated by different lysis techniques. The spectra 1232 and 1234 were collected from captured cellular components obtained by the chemical lysis procedure explained above. The spectrum 1236 was collected from captured cellular components obtained by the electrical lysis procedure explained above. The spectrum 1238 was collected from captured cellular components obtained by the glycerol based lysis procedure explained above. The spectra 1232, 1234, 1236 and 1238 were acquired in the positive ion mode. The output represented by the spectrum 1232 has an absolute signal intensity of 2.9E5. The output represented by the spectrum 1204 has an absolute signal intensity of 1.4E6. The output represented by the spectrum 1206 has an absolute signal intensity of 1.6E6. The output represented by the spectrum 1208 has an absolute signal intensity of 1.1E6. The laser power setting was 15 uJ with 3 shots/scan and 1 scan/step. The scan range was m/z 100-1500 and the raster step size was 50 um.

FIG. 12E shows four different mass spectra 1242, 1244, 1246 and 1248 from analysis of cellular components isolated by different lysis techniques. The spectra 1242 and 1244 were collected from captured cellular components obtained by the chemical lysis procedure explained above. The spectrum 1246 was collected from captured cellular components obtained by the electrical lysis procedure explained above. The spectrum 1248 was collected from captured cellular components obtained by the glycerol based lysis procedure explained above. The spectra 1242, 1244, 1246 and 1248 were acquired in the positive ion mode. The absolute signal intensity for the outputs in FIG. 12E was 3.5E5. The laser power setting was 15 uJ with 3 shots/scan and 1 scan/step. The scan range was m/z 150-200 and the raster step size was 50 um. As may be seen in FIG. 12E, peaks representing Pyroglutamic acid (PGA), Phosphocholine, and 5-Hydroxymethyluracil (5-HMU) were discovered. FIG. 12E shows a signal at m/z 184.0739 (Phosphocholine) that may be indicative of a lipid headgroup from the membranes of captured nuclei.

FIG. 12F shows four different mass spectra 1252, 1254, 1256 and 1258 from analysis of cellular components isolated by different lysis techniques. The spectra 1252 and 1254 were collected from captured cellular components obtained by the chemical lysis procedure explained above. The spectrum 1256 was collected from captured cellular components obtained by the electrical lysis procedure explained above. The spectrum 1258 was collected from captured cellular components obtained by the glycerol based lysis procedure explained above. The spectra 1252, 1254, 1256 and 1258 were acquired in the positive ion mode. The absolute signal intensity for the outputs in FIG. 12F was 2E5. The laser power setting was 15 uJ with 3 shots/scan and 1 scan/step. The scan range was m/z 550-850 and the raster step size was 50 um. Unlike the spectra in FIG. 12E, membrane lipids were not observed in the spectra in FIG. 12F.

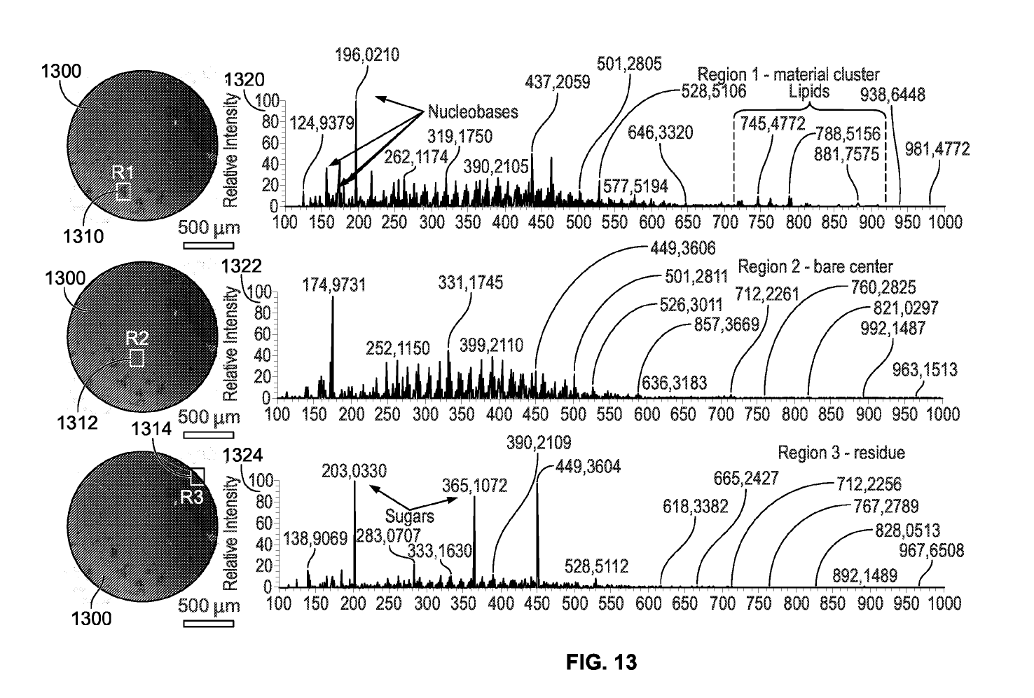

FIG. 13 shows averaged mass spectra from different regions of a nanopost array partly covered with captured cellular material. A bright field image 1300 shows clusters of cellular components that have been captured on the nanopost array by the conjugated anti-Nup98 antibody. A first region of interest 1310 includes a cluster of captured nuclear material, a second region of interest 1312 includes a region of the array that appears free of deposited material and a third region of interest 1314 includes residue from the nuclear suspension buffer. An output graph 1320 shows the averaged mass spectrum over the first region of interest. An output graph 1322 shows the averaged mass spectrum over the second region of interest. An output graph 1324 shows the averaged mass spectrum over the third region of interest. The spectra 1320, 1322 and 1324 show distinct spectral patterns.

Unique mass spectra are obtained from specific regions of the nanopost array, including: the first region 1310 (R1), a cluster of captured nuclear material; the second region 1312 (R2), a region of the chip apparently free of deposited material; and the third region 1314 (R3), a region containing residual material from nuclear suspension. As shown in the graph 1320, Region 1 exhibits signals corresponding to nucleobases such as adenine, guanine, cytosine, and thymine, as well as membrane lipids such as phosphatidylethanolamines. As shown in the graph 1322, Region 2 is dominated by signals that are suspected to arise from fragments of the capture antibody that are generated on ablation of the surface material. As shown in graph 1324, Region 3 shows significant signal for mono- and disaccharides.

The spectrum 1324 shows background signal that is suspected to arise from the fragments of the capture antibody that are generated on ablation of the surface material in the second region 1314 that does not show captured material. The spectrum 1326 shows signals arising from nucleus suspension buffer residue such as mono- and disaccharides for the third region 1316 near the periphery. The presence of these components is likely due to incomplete removal of the nucleus suspension buffer used in the lysis process.

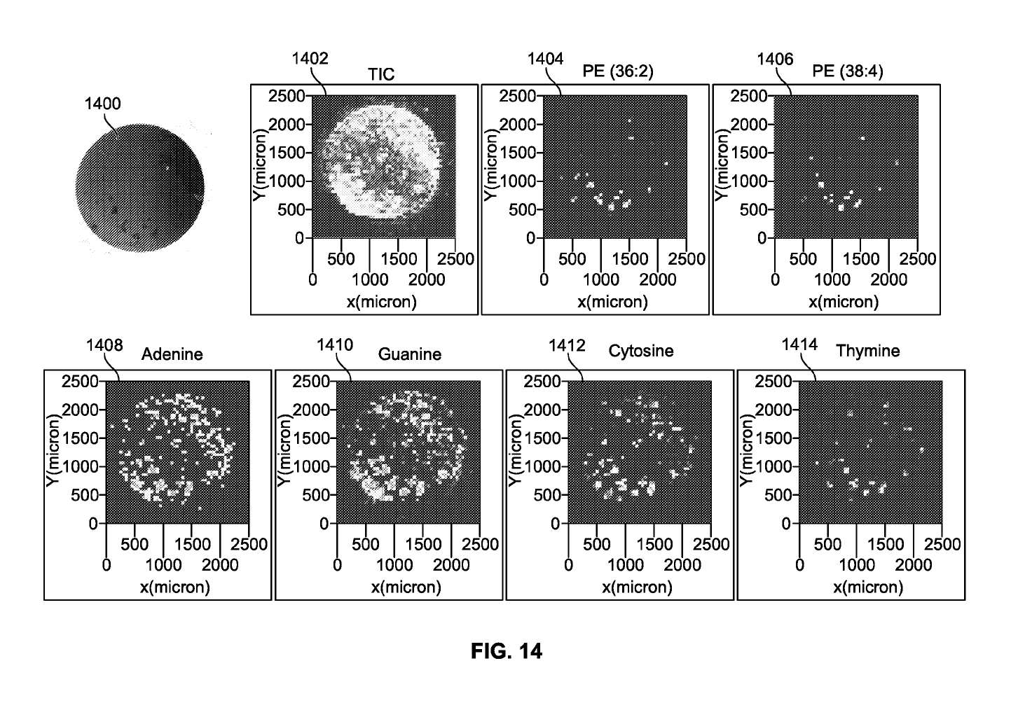

FIG. 14 shows a series of reconstructed ion images that show metabolite ion signals colocalized with the captured material as observed by microscopy. The images in FIG. 14 are reconstructed ion images showing spatial distribution of ion signals over the surface of an anti-Nup98-functionalized nanopost array following capture of HepG2/C3A nuclei. The imaging operational mode of the instrument control software allows for the acquisition of spectra from an array of distinct spatial positions over the surface of the sample. At each position, a mass spectrum is recorded. From these spectra and their corresponding x-y positions on the sample, it is possible to plot the intensity of an observed ion signal as a function of location on the sample surface. In the case of the captured cellular material on the nanopost surface in FIG. 14, lipid and nucleobase MS signals are highly colocalized with one another and with the regions of the optical image where captured material is visible.

FIG. 14 includes an image 1400 that shows the nanopost array with the captured material. An ion image 1402 is the total ion count (TIC) of all the intensities. The TIC represents the sum of all ion intensities in the mass spectrum for a given x-y pixel. FIG. 14 includes other ion images 1404, 1406, 1408, 1410, 1412 and 1414. Each of these other ion images show the intensity distributions for a single m/z value assigned to a metabolite ion. All metabolite ion assignments were made based on accurate mass measurements with an error of less than 5 ppm, and ion images were generated with a m/z window of ±0.005. All metabolites assigned below are detected as the [M+2Na−H]⁺ species. The ion image 1404 is generated from the signal at m/z 788.5156, assigned as the membrane lipid phosphatidylethanolamine (PE) with the given total fatty acid composition (36 carbons:2 double bonds). The ion image 1406 is generated from the signal at m/z 812.5171, assigned as the membrane lipid phosphatidylethanolamine (PE) with the given total fatty acid composition (38 carbons:4 double bonds).

The image 1408 is generated from the signal at m/z 180.0260, assigned as the nucleobase adenine. The image 1410 is generated from the signal at m/z 156.0148, assigned as the nucleobase guanine. The image 1412 is generated from the signal at m/z 156.0148, assigned as the nucleobase cytosine. The image 1414 is generated from the signal at m/z 171.0142, assigned as the nucleobase thymine. Adenine, guanine, cytosine, and thymine are all integral to the structure of nucleic acids and many other metabolites.

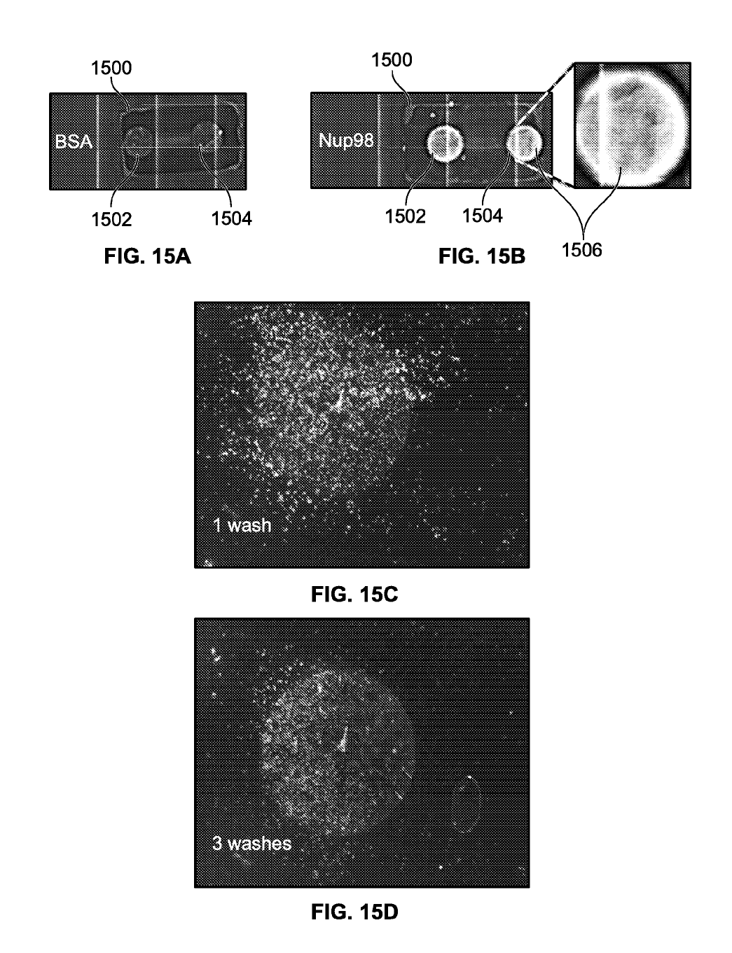

FIGS. 15A-15B are fluorescent images of NAPA chips during the above described process. Specifically FIG. 15A is a fluorescent image of a BSA control NAPA chip and FIG. 15B is a fluorescent image of a Nup98-coated NAPA chip 1500 following staining with a fluorescence-conjugated secondary antibody that recognizes the Nup98 antibody. NAPA-patterned areas are visible as two lighter circles 1502 and 1504 in each image; unpatterned silicon is visible as a darker rectangular area around the patterned areas. Colored (yellow) areas 1506 represents fluorescence from a secondary antibody adhered onto the Nup98 antibody deposited on the NAPA patterned areas 1502 and 1504.

FIGS. 15C-15D are fluorescent images obtained using a 4× objective of propidium iodide stained cell nuclei attached onto a patterned NAPA surface following nucleus deposition and washing. FIG. 15C is a fluorescent image obtained after one wash while FIG. 15D is a fluorescent image obtained after three washes. The NAPA surface was conjugated with anti-Nup98 antibody prior to the addition of nuclei. Propidium iodide stains the DNA in the nucleus. Nuclei are primarily attached to the NAPA area (NAPA-patterned areas are visible as circles in each image in FIGS. 15C-15D and unpatterned silicon is visible as the darker area around the patterned areas). As may be seen in FIGS. 15C-15D, non-specific association of nuclei on the unpatterned silicon is reduced by washing.

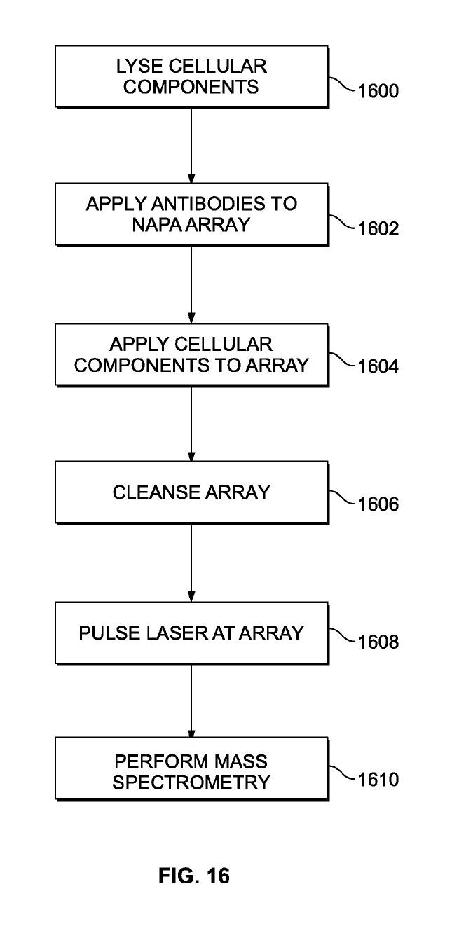

FIG. 16 is a flow diagram of the process of laser desorption ionization of cellular components for mass spectrometry analysis of biomolecules by the system 100 in FIG. 1. A cell sample is either suspended intact or lysed via a chemical or electrical process (1600). An antibody composition is conjugated to a columnar array such as the nanopost array 116 in FIG. 1 (1602). The intact or lysed cell sample for analysis is deposited onto the columnar array (1604). The antibody selectively captures cells or cellular components by binding to receptors on the surface of the deposited components. The array 116 is then cleansed to isolate the captured components and remove nonspecifically bound material (1606). The laser 110 is activated to desorb and ionize the components of the cell sample on the array 116 (1608). The mass spectrometer is used to perform chemical analysis of the ionized cell components and output a spectrum (1610).

The above described system and process allows cells or cell compartments to be captured and analyzed directly on the same surface. This minimizes processing steps, and thereby reduces the potential for degradation of biomolecules or introduction of interferences. The streamlined workflow also reduces the time and labor required for analysis of samples.

The LDI-MS technique involves applying antibodies to the silicon nanopost arrays to hold different cells. The chemistry used for functionalization of NAPA chips with antibodies or other conjugates can be applied for selective capture of different cell compartments or intact cells, permitting selective analysis of biomolecules with high biological specificity.

Proof of concept experiments have demonstrated capture of nuclei on NAPA chips, as confirmed by bright-field, fluorescence, and scanning electron microscopies. LDI-MS analysis of these captured nuclei has detected signals assigned to DNA- and RNA-base metabolites (thymine, adenine, and guanine) and phosphatidylcholine and phosphatidylethanolamine membrane lipids.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

1. A system to analyze a biomolecular sample, the system comprising:
a columnar array having a plurality of columnar members;
an antibody attached to the columnar array;
a laser focused at a sample captured by the antibody on the columnar array, the laser operable to apply a pulse to desorb and ionize the sample;
a mass spectrometer; and
a controller coupled to the mass spectrometer to analyze the output based on the detected ionized component from the columnar array.

2. The system of claim 1, further comprising an electrical lysing chip, the lysing chip operable to lyse a cell component.

3. The system of claim 1, wherein the sample is a cellular component lysed from a cell.

4. The system of claim 3, wherein the cellular component is a nucleus.

5. The system of claim 3, wherein the cellular component is chemically lysed.

6. The system of claim 1, wherein the antibody is selected to capture a specific cell or cellular component.

7. The system of claim 6, wherein the antibody is an antibody against Nup98.

8. The system of claim 1, wherein the columnar array is a silicon nanopost array.

9. The system of claim 1, wherein the columnar array is a laser-induced silicon microcolumn array.

10. A method to analyze a biomolecular sample, the method comprising:
attaching an antibody to a columnar array;
placing a sample for analysis on the columnar array so the antibody selectively captures the sample;
activating a laser to desorb and ionize the sample; and
performing mass spectrometry on the ionized sample.

11. The method of claim 10, further comprising lysing a cell sample, wherein the resulting sample is a cellular component of the cell sample;

12. The method of claim 11, further comprising washing the lysed cell sample prior to activating the laser.

13. The method of claim 11, wherein the lysing is performed by chemical lysing.

14. The method of claim 11, wherein the lysing is performed electrically.

15. The method of claim 11, wherein the cellular component is a nucleus of the cell sample.

16. The method of claim 10, further comprising selecting the antibody to selectively capture a cell or a cellular component.

17. The method of claim 11, wherein the antibody is an antibody against Nup98.

18. The method of claim 11, wherein the columnar array is a silicon nanopost array.

19. The method of claim 11, wherein the columnar array is a laser-induced silicon microcolumn array.

20. A laser desorption ionization mass spectrometry system for analyzing biomolecules comprising:
a silicon nanopost array having a plurality of nanoposts;
an antibody against Nup98 attached to the columnar array;
a laser focused at a cellular component sample obtained by a lysed cell, the cellular component sample captured by the antibody on the nanopost array, the laser operable to apply a pulse to desorb and ionize the cellular component sample;
a mass spectrometer; and
a controller coupled to the mass spectrometer to analyze the output based on the detected ionized cellular component sample captured on the nanopost array.
